(19) United States
(12) Patent Application Publication
(10) Pub. No.: US 2015/0258660 A1
(43) Pub. Date: Sep. 17, 2015

(54) POLYUREA-BASED MATERIAL, POLISHING AND GRINDING MEDIA INCLUDING THE POLYUREA-BASED MATERIAL, AND METHODS OF FORMING AND USING SAME

(71) Applicant: JH Rhodes Company, Inc., Tempe, AZ (US)

(72) Inventors: George James Wasilczyk, Allentown, PA (US); Scott Daskiewich, Oriskany, NY (US)

(21) Appl. No.: 14/645,150

(22) Filed: Mar. 11, 2015

Related U.S. Application Data
(60) Provisional application No. 61/953,440, filed on Mar. 14, 2014.

(51) Int. Cl. B24D 3/28; C07C 275/14; B24D 3/32; C08G 18/32

(52) U.S. Cl. CPC B24D 3/28; C08G 18/3228; C07C 275/14; B24D 3/32

(57) ABSTRACT

Polyurea-based materials suitable for grinding media and polishing media, media including the polyurea-based material, and methods of forming and using the polyurea-based material and media are disclosed. The polyurea-based material is formed using one or more secondary polyamines, which allows for slower reaction rates and produces material having desired properties.

### CROSS REFERENCE TO RELATED APPLICATIONS

This application claims the benefit of U.S. Provisional Application No. 61/953,440, entitled POLYUREA-BASED MATERIAL, POLISHING AND GRINDING MEDIA INCLUDING THE POLYUREA-BASED MATERIAL, AND METHODS OF FORMING AND USING SAME, and filed Mar. 14, 2014, the contents of which are hereby incorporated herein by reference to the extent such contents do not conflict with the present disclosure.

### FIELD OF INVENTION

The present disclosure generally relates to polyurea-based materials, to grinding and polishing media including the polyurea-based materials, and to methods of forming and using the polyurea-based materials and the grinding and polishing media. More particularly, the disclosure relates to polyurea-based materials formed using secondary polyamines, to grinding and polishing media including such polyurea-based materials, and to methods of forming and using the materials and media.

### BACKGROUND OF THE DISCLOSURE

Grinding and polishing media, such as grinding wheels and polishing pads, are useful in many applications. For example, grinding and/or polishing media can be used for edge grinding substrates, such as glass, and for polishing substrates, such as sapphire substrates or semiconductor wafers. To polish or grind a surface of a substrate, the grinding or polishing media is placed adjacent to the substrate and moved relative to the substrate surface. This relative movement can be created by rotating the media, by rotating the substrate, by orbital movement of the substrate or media, or a combination of such movements. Additionally or alternatively, linear or any other useful relative motion between the media and the substrate can be used. A force can be applied to press the media against the substrate surface. The grinding or polishing can be performed to varying degrees, such as to remove larger imperfections, to achieve a mirror finish, to obtain a desired final flatness, or the like.

Grinding and polishing media often include elastomeric material, such as polyurethane, polyurea, and polyurethane-polyurea hybrid materials. In the case of grinding media, the elastomeric material can be used to bind abrasive materials. For polishing media, the elastomeric material can form a foam matrix that is used as a polishing pad. Each of the various elastomeric materials has various advantages and drawbacks.

In the common use of an elastomeric grinding or polishing medium, the medium may be subjected to relatively high temperatures, arising from friction between the medium and the substrate. The relatively high temperature can cause properties of the medium to change, such that the medium is not as effective as it was at lower temperature. For example, the medium can have an unstable (i.e., variable and/or unpredictable) polishing or grinding rate over the life of the medium. The polishing or grinding rate can vary, for example, +/−8% or greater of the nominal polishing or grinding rate. Additionally or alternatively, non-uniformity of removal rates across a surface of the substrate can increase at elevated temperatures. The non-uniformity in turn, can cause planarity problems wherein planarity values exceed specifications for example.

The decline in performance of the medium can be attributed to several factors in the design of the grinding or polishing medium, such as the relative strength of the bonds connecting a hard segment (e.g., polyisocyanate) to the soft segment (e.g., polyol). With typical polyurethane or polyurethane-polyurea hybrid grinding or polishing media, a decline in performance often occurs with high temperature applications, such as high speed edge grinding or sapphire polishing. Typical polyurethane or polyurethane-polyurea hybrid grinding wheels or foams can experience a significant softening of their physical properties that results in performance declines. The softening of a grinding wheel or polishing pad results in a reduction of substrate stock removal, an increase in flatness variation, edge roll off, and a reduction in the life of the medium.

Polyurea produced from the reaction of primary polyamines with a polyisocyanate can exhibit more desirable properties such as relatively stable (e.g., relatively non-variable and/or predictable) material removal rates, relatively stable removal rate uniformity, and relatively high longevity, relative to polyurethane or polyurethane-polyurea hybrid media, particularly in high temperature applications. However, primary polyamines are costly relative to standard polyurethane raw materials; thus, media formed with primary amine starting material are relatively expensive. In addition, primary amines have high reactivity rates with polyisocyanates, making formation of media with desired properties difficult to control.

Accordingly, improved polishing and grinding media with improved performance (e.g., relatively stable and uniform removal rates) and relatively high longevity that are formed using reactants with reaction rates that allow for desired control of media properties are desired.

### SUMMARY OF THE DISCLOSURE

Various embodiments of the present disclosure relate to improved materials suitable for media for polishing and/or grinding a surface of a substrate. While the ways in which various embodiments of the present disclosure address drawbacks of prior polishing and grinding materials and media are discussed in more detail below, in general, various embodiments of the disclosure provide a polyurea-based material formed using a secondary amine. Using secondary polyamines rather than primary polyamines is advantageous because polishing and grinding media including such materials can be formed at a lower cost (e.g., about 25 to 50% lower), with controllable reaction rates (e.g., up to about 10 times slower), which allows formation of polyurea-based polishing and grinding media with desired properties.

In accordance with exemplary embodiments of the disclosure, a polyurea-based material for grinding or polishing a surface of a substrate includes a polyurea, wherein the polyurea is formed from one or more secondary polyamines having a general formula of R[—NH—R′]ₙ, wherein n is greater than or equal to 2 and wherein R is not H and R′ is not H, and one or more of polyisocyanates, polyisocyanate derivatives, and polyisocyanate products. In accordance with various exemplary aspects, the polyurea can be formed with a catalyst, and in accordance with other aspects, the polyurea can be formed without a catalyst. The polyurea can have the general formula of

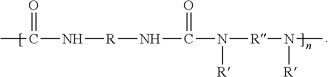

The polyurea-based material can comprise, for example, about 5 to about 80 wt %, or 100 wt % polyurea. In accordance with various aspects of the disclosure, a molecular weight of the polyurea is between about 50 and about 6000 Da, between about 250 and about 6000 Da, or between about 250 and about 3000 Da. In accordance with further exemplary aspects, the polyurea-based material further comprises a chain extender, such as one or more of a secondary polyamine chain extender, a polyisocyanate, and a secondary amine polyol. Additionally or alternatively, the polyurea-based material can include a cell stabilizer. The polyurea-based material can also include from 0 wt % to about 80 wt % organic and/or inorganic filler. The polyurea-based material can optionally be formed using a foaming agent. In accordance with further examples, a bulk density of the polyurea-based material is between about 0.2 g/cm³ and 1.2 g/cm³. A Shore A hardness of the polyurea-based materials can be between about 10 and 95. The polyurea-based material can include grooves on a surface to facilitate polishing or grinding. Additionally or alternatively, the polyurea-based material can include adhesive on a surface to facilitate attachment of the polyurea-based material to a polishing or grinding machine. The polyurea-based material can be formed into a grinding wheel or a pad. In the case of a pad, the pad can be singular or part of a stacked pad.

In accordance with additional exemplary embodiments of the disclosure, a method of forming a polyurea-based material includes the steps of mixing one or more secondary polyamines having a general formula of R[—NH—R′]ₙ, wherein n is greater than or equal to 2 and wherein R is not H and R′ is not H. Exemplary secondary polyamines can be either low molecular weight chain extenders (e.g., having a molecular weight between about 50 and 200 Da) or long chain polyols (e.g., having a molecular weight between about 200 and 3000 Da). The method further includes reacting the one or more secondary polyamines with one or more of polyisocyanates, polyisocyanate derivatives, and polyisocyanate products to form a polyurea-based composition. Exemplary methods also include pouring the polyurea-based composition into a mold. In accordance with various aspects of these embodiments, the method includes a step of mixing the one or more secondary polyamines within a foaming agent. In accordance with further aspects, the method includes a step of mixing one or more blowing agents, such as water, with the one or more secondary polyamines. Exemplary methods can also include mixing one or more surfactants with the secondary polyamine. The method can also include a step of curing the polyurea-based composition—e.g., at a temperature of about 100° C. to about 130° C. for about 6 hours to about 12 hours. Exemplary methods can also include a step of machining, forming grooves into a surface of the polyurea-based material, skiving the polyurea-based material, and/or adding adhesive to a surface of the polyurea-based material.

In accordance with yet additional exemplary embodiments of the disclosure, a polishing media (e.g., a polishing pad) comprises a polyurea-based material as described herein. The polishing media can include grooves on a surface. Additionally or alternatively, the polishing media can include adhesive material on a surface.

In accordance with yet further exemplary embodiments of the disclosure, a method of forming a polishing media includes a method of forming a polyurea-based material as described herein. The method can additionally include steps of skiving the polyurea-based material, forming grooves in the polyurea-based material, and/or adding adhesive material to a surface of the polyurea-based material.

In accordance with yet additional exemplary embodiments of the disclosure, a method of forming a grinding media includes a method of forming a polyurea-based material as described herein. The method can additionally include steps of machining the polyurea-based material—e.g., to form a grinding wheel.

### BRIEF DESCRIPTION OF THE DRAWING FIGURES

A more complete understanding of the embodiments of the present disclosure may be derived by referring to the detailed description and claims when considered in connection with the following illustrative figures.

FIG. 1 illustrates a general formula of polyurea.

FIG. 2(A) illustrates a portion of a grinding apparatus in accordance with exemplary embodiments of the disclosure.

FIG. 2(B) illustrates a plan view of a grinding media in accordance with further exemplary embodiments of the disclosure.

FIG. 3 illustrates another exemplary grinding apparatus in accordance with various exemplary embodiments of the disclosure.

FIGS. 4(A), 4(B) and 4(C) illustrate a grinding ring in accordance with additional exemplary embodiments of the disclosure.

FIG. 5 illustrates a portion of an exemplary polishing medium in accordance with yet additional exemplary embodiments of the disclosure.

It will be appreciated that elements in the figures are illustrated for simplicity and clarity and have not necessarily been drawn to scale. For example, the dimensions of some of the elements in the figures may be exaggerated relative to other elements to help to improve understanding of illustrated embodiments of the present disclosure.

### DETAILED DESCRIPTION OF EXEMPLARY EMBODIMENTS OF THE INVENTION

The description of exemplary embodiments of polyurea-based materials, media, such as polishing pads and grinding wheels, including the polyurea-based materials, methods of forming and using the polyurea-based materials and media provided below is merely exemplary and is intended for purposes of illustration only; the following description is not intended to limit the scope of the disclosure or the claims. Moreover, recitation of multiple embodiments having stated features, compositions, or properties is not intended to exclude other embodiments having additional features, compositions, or properties, or other embodiments incorporating different combinations of the stated features, compositions, or properties.

The materials and media described herein can be used for a variety of applications, including polishing or grinding a surface of a substrate. By way of examples, the materials and media can be used to polish or grind a surface of glass, semiconductor material (e.g., silicon), sapphire, or similar materials. Exemplary media exhibit relatively high thermal stability and are relatively easy to form, compared to traditional media used for such applications.

In accordance with various embodiments of the disclosure, a polyurea-based material comprises polyurea, wherein the polyurea is formed from one or more secondary polyamines and one or more of polyisocyanates, polyisocyanate derivatives, and polyisocyanate products.

The polyurea-based material can be used to form polishing media (e.g., pads) and/or grinding media (e.g., grinding wheels) that are used to remove material from a surface of a substrate.

The polyurea-based materials and media (e.g., polishing pads, grinding wheels, and the like) described herein are advantageous over conventional polyurea, polyurethane, and polyurethane-polyurea hybrid materials, because the starting materials (e.g., the secondary amine(s)) for the polyurea-based materials are less expensive, relative to conventional polyurea materials. In addition, the polyurea-based materials described herein perform better with respect to thermal stability—i.e., the performance of the polyurea-based materials is relatively stable-compared to polyurethane and polyurethane-polyurea hybrid materials. Specifically, glass transition temperature of exemplary polyurea materials described herein can be increased by up to 40° C. compared to conventional polyurethane or polyurethane-polyurea hybrid materials. Increasing the glass transition temperature increases the temperature range in which the medium remains in an elastic state and/or can withstand embrittlement.

The polyurea-based materials and media described herein are also advantageous over polyurea-based materials formed using primary amines, because reaction rates of secondary polyamines with a polyisocyanate or derivative or product thereof are slower relative to reactions between primary amines and a polyisocyanate or a derivative or product thereof, allowing slower and more controlled reactions (e.g., up to 10 times slower) for formation of the polyurea-based material. The slower reaction rate allows polyurea-based material and media including the polyurea-based material to be formed with desired properties, such as those set forth below. In addition, secondary amines tend to be less expensive than primary amines by, e.g., about 25% to about 50%. Thus, polyurea-based material and media formed with the polyurea-based material is less expensive than similar materials and media formed using primary amines.

Exemplary secondary polyamines suitable for forming a polyurea-based material as described herein can include one or more of: polyether diamines, polycarbonate diamines, polyester diamines and polycaprolactone diamines. An exemplary secondary diamine is available from Huntsman under the name SD-2001 polyether. Various exemplary polyamines suitable for use to form the polyurea-based material includes a general formula: R[—NH—R′]ₙ, wherein n is greater than or equal to 2, and wherein R is not H and R′ is not H.

By way of examples, the secondary polyamines can include the formula above, where R is a polyoxypropylene polyether, R′ is an isobutyl group and n is 2.

A molecular weight of the secondary amine can range from about 50 to about 6000 Da, about 250 to about 6000 Da, about 250 to about 3000 Da, or about 500 to about 3000 Da, as determined by gel permeation chromatography (GPC) using polystyrene standards.

Exemplary polyisocyanates or derivatives or products thereof that can be used to prepare the polyurea-based material include, but are not limited to: one or more aromatic polyisocyanates, such as toluene diisocyanate (TDI), methylene diphenyl diisocyanate (MDI)—e.g., 2,2′-MDI, 2,4′-MDI, or 4,4′-MDI, or their derivatives; aliphatic polyisocyanates; ethylenically unsaturated polyisocyanates; alicyclic polyisocyanates; aromatic polyisocyanates wherein the isocyanate groups are not bonded directly to the aromatic ring, e.g., xylene diisocyanate; aromatic polyisocyanates wherein the isocyanate groups are bonded directly to the aromatic ring, e.g., benzene diisocyanate; halogenated, alkylated, alkoxylated, nitrated, carbodiimide modified, urea modified and biuret modified derivatives of polyisocyanates belonging to these classes; and dimerized and trimerized products of polyisocyanates belonging to these classes, and any combination of such polyisocyanates or derivatives or products thereof.

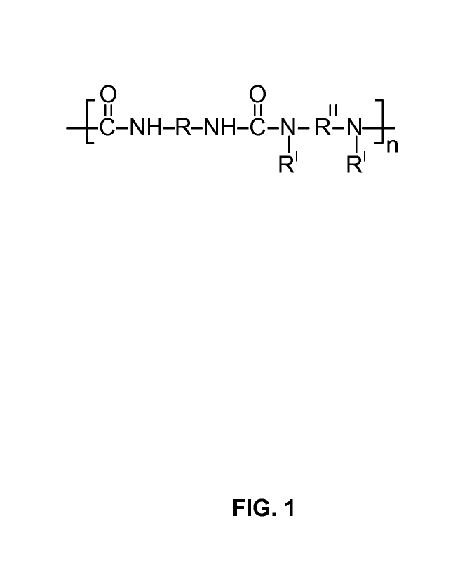

The polyurea resulting from a reaction between a secondary amine and one or more of polyisocyanates, polyisocyanate derivatives, and polyisocyanate products can have a general formula illustrated in FIG. 1. Exemplary examples of R and R′ are where R is a polyoxypropylene polyether and R′ is an isobutyl group. Additionally, R and R′ can be alkane, alkyne, alkene, polyether, polyester, polycaprolactone, polycarbonate branched or unbranched polymers or co-polymers with or without aromatic groups. R″ can include the same or similar compounds as R and/or R′.

The polyurea-based material can also include additional materials, such as cell stabilizers, cell openers, chain extenders, fillers, surfactants, foaming agents, and blowing agents.

Exemplary cell stabilizers include Momentive brand L-6100 and Dow Corning DC-193.

Cell openers can promote cell opening during the interaction of two cells in the liquid phase. Exemplary cell openers include, but are not limited to, non-hydrolizable polydimethylsiloxanes, polyalkyleoxides, dimethylsiloxy, methylpolyethersiloxy, silicone copolymers, such as Dabco DC-3043 or Dabco DC-3042, available from Air Products, Allentown, Pa.

Secondary amine chain extenders can also be used in conjunction with secondary amine polyols and polyisocyanate functional derivatives to produce polyurea. Examples of secondary amine chain extenders include Ethacure 90 and 420, available from Albemarle Corporation, and hydrogenated butyl MDA.

Fillers can include organic and/or inorganic fillers. Exemplary inorganic fillers include abrading particles that include, but are not limited to, particles of, for example, cerium oxides, silicon oxides, aluminum oxides, zirconia, iron oxides, manganese dioxides, kaolin clays, montmorillonite clays, titanium oxides, silicon carbides and diamond. A size of the inorganic particles can range from about 0.001 to about 1000 microns, or about 0.5 to about 3.0 microns average diameter. Exemplary organic fillers include polyurethane foam, epoxy, polystyrene, polyacrylic, polyimide, or other thermoplastic or thermoset materials.

Foaming agents can facilitate cell growth. Exemplary foaming agents include one or more of a hydroflourocarbon (HFC) or azeotrope of 2 or more hydrocarbon (HFCs), such as 1,1,1,3,3-pentaflourobutane (HFC-365), 1,1,1,2-tetraflouroethane (HFC-134a), methoxy-nonafluorobutane (HFE-7100) and a free radical initiator comprising an azonitrile, such as 2,4-Dimethyl, 2,2′-Azobis Pentanenitrile. Particular foaming agents include the HFCs Solkane® 365mfc and 134a (Solvay, Hannover, Germany), and free radical initiators Vazo 52 (DuPont, Wilmington, Del.). Water is also commonly used to foam polyurea systems via the reaction between water and isocyanate, thereby producing carbon dioxide. Various combinations of foaming agents, including, but not limited to those disclosed herein, can be incorporated into a polyurea-based material or media including the material and are contemplated in this disclosure.

Exemplary surfactants include Air Products DC-2525, DC-5000, and DC-5357.

Exemplary polyurea-based material can include a foam matrix. The foam matrix can have a bulk density of 0.2 to 1.2 g/cm³ and/or a hardness of 10 to 95 Shore A. An exemplary range of bulk density is from about 0.35 to 0.65 g/cm³. An exemplary range for hardness is from about 50 to 85 Shore A.

Grinding Media

Generally, grinding media, such as grinding wheels, are made by mixing, casting and hardening by adding filler particles and, if desired, one or more foaming agents, and optionally one or more color pigments and/or other optional materials during polyurea-based material formation as described herein.

Grinding media as described herein exhibit improved properties relative to prior grinding media by using the polyurea-based material as described herein as a binding material. Prior grinding media often used a rigid binding material, such as phenol or epoxy, which often degrades too quickly or produces a rough finish in fine grinding applications, because of the lack of elasticity. To offer more elasticity, polyurethane and polyurethane-polyurea hybrid grinding media were developed, but such media hold filler particles weakly and also change shape and properties during processing, resulting in relatively poor performance of the materials. Because the glass transition temperature of polyurethane is low (typically from about 100° C. to 140° C.), cooling via wet grinding is often used to keep a process temperature lower than the softening temperature; this reduces productivity of process using such media and increases costs associated with use of the media.

Although binding materials for filler particles desirably have a strong bonding strength of abrasive grains, good elasticity, and heat resistance, conventional polyurethane and polyurethane-polyurea hybrid grinding wheels lack these characteristics, and grinding wheels with satisfactory performance characteristics such as heat resistance, have not been obtained. Grinding wheels with satisfactory performance have been created using polyurea foams formed from primary amines, but cost of the primary polyamine raw materials as well as extremely fast reactivity times prevent this technology from being widely accepted or practical. Exemplary polyurea-based materials described herein solve the above problems with grinding media that includes a foam matrix of polyurea-based material formed from the reaction of a secondary polyamine and polyisocyanates, polyisocyanate derivatives, or polyisocyanate products. This combination of ingredients provides advantages over polyurethane media in thermal performance and over polyurea media formed with primary amines in cost and reactivity rate. Also, polyurea glass transition temperatures up to 160° C. of polyurea-based material as described herein can be obtained with secondary amine polyurea.

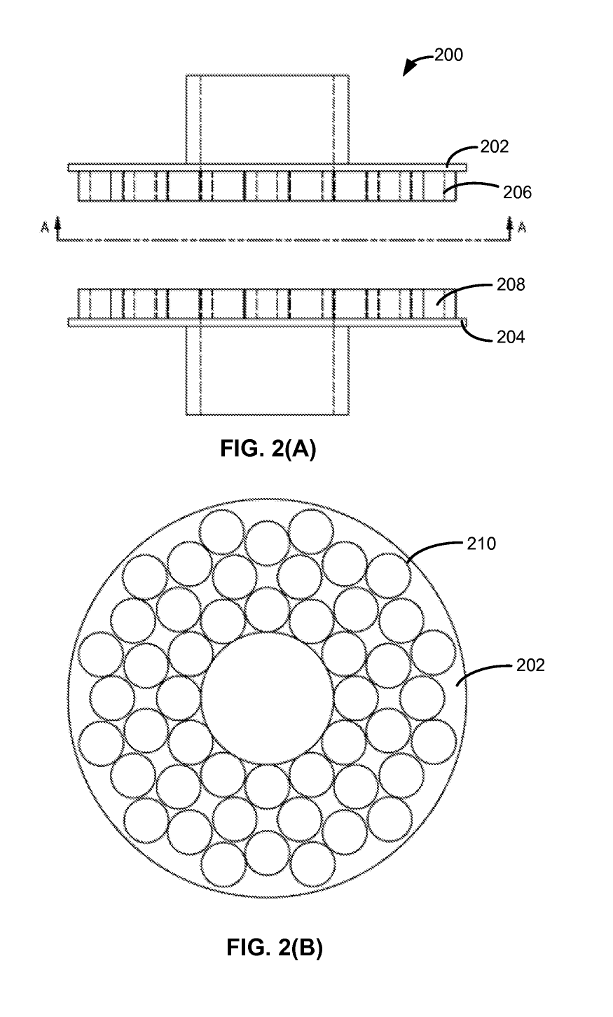

FIGS. 2(A)-4(C) illustrate a grinding apparatus/systems 200, 300 and grinding media 206, 208 in accordance with exemplary embodiments of the disclosure. With reference to FIG. 2(a), double-sided grinding apparatus 200 is illustrated. Grinding apparatus 200 includes a top platen 202, having grinding media 206 attached thereto, and platen 204, having grinding media 208 attached thereto. Material can be removed from a top and bottom surface of a substrate (not illustrated), by moving grinding media 206 and 208 relative to the top and bottom surfaces. The movement of the media/platens and/or of the substrate can be, for example, rotational, orbital, and/or linear. One or both platens 202, 204 can move during a grinding process.

FIG. 2(B) illustrates a plan view, along line A-A of FIG. 2(A), of grinding media 206 and platen 202. As illustrated, grinding media 206 can comprise one or more (e.g., a plurality of) tubular members 210. The number and size of tubular members 210 can vary according to application. By way of one example, apparatus can include about 50 tubular members on platen 202, wherein the tubular members have an outer diameter of about 2 to about 20 cm, an inner diameter of about 0 to about 10 cm, and an initial height of about 1.3 to about 40 mm. Platen 204 and media 208 can be the same or similar to platen 202 and media 206.

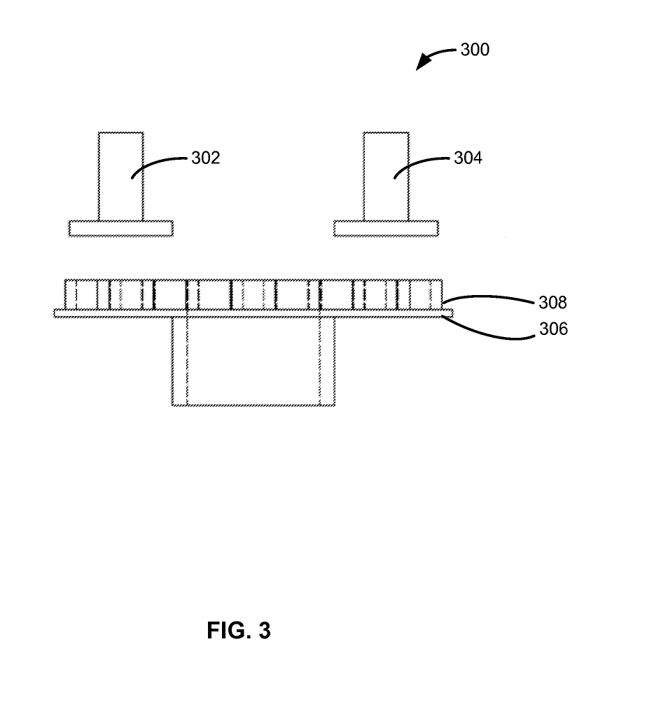

FIG. 3 illustrates single-sided grinding apparatus 300 in accordance with further exemplary embodiments of the disclosure. Apparatus 300 includes supports 302, 304, a platen 306 and grinding media 308. Platen 306 and media 308 can be the same or similar to platens 202, 204 and media 206, 208.

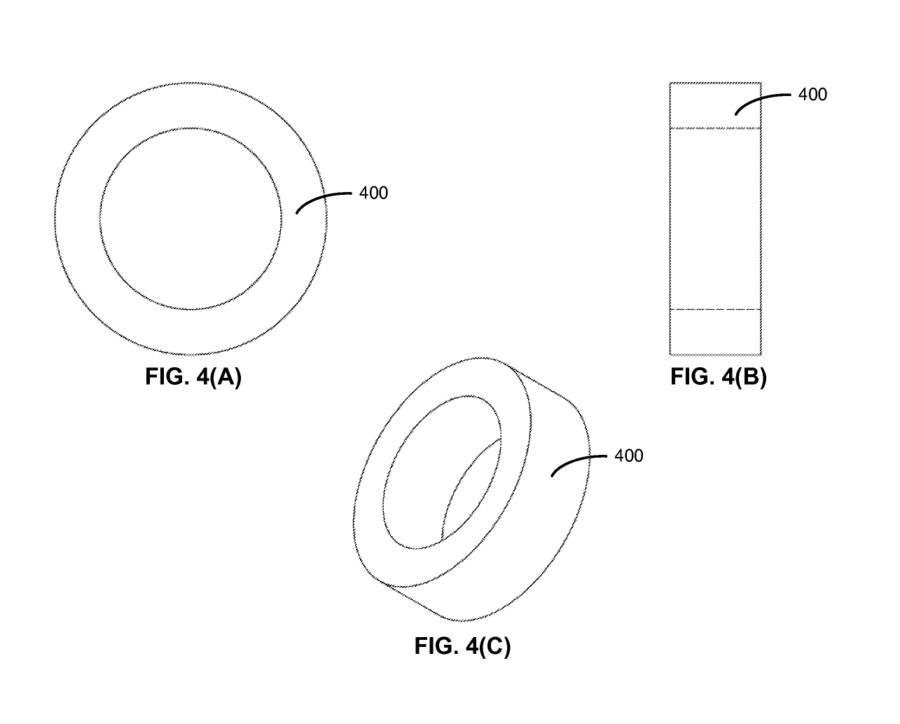

FIGS. 4(A)-4(C) illustrate a tubular member (i.e., a grinding stone) 400 in accordance with exemplary embodiments. Tubular member 400 can be the same or similar to tubular member 210.

Exemplary grinding media (e.g., tubular members 210, 400) include about 5 to about 80 wt % polyurea material and about 0 to about 80 wt % filler/filler particles.

Polishing Media

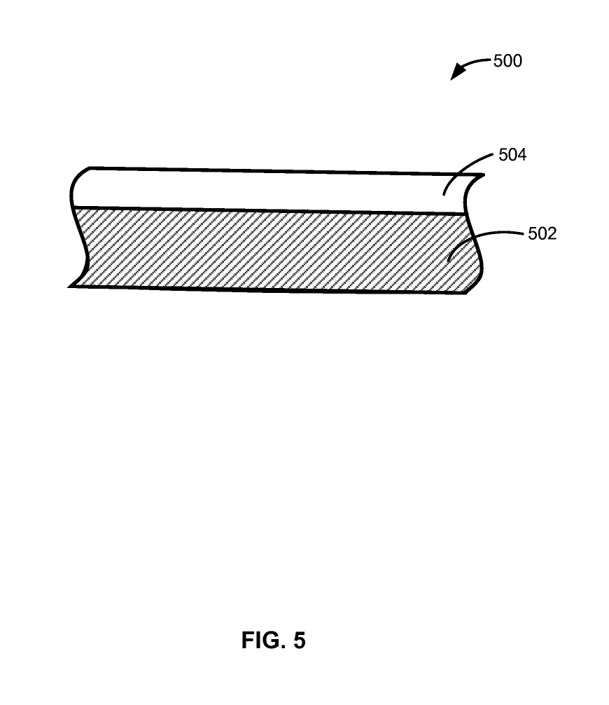

Polishing media can include a foam material comprised of a polyurea-based material as described herein. FIG. 5 illustrates a portion 500 of an exemplary polishing medium in accordance with exemplary embodiments of the disclosure. In the illustrative example, portion 500 includes a polishing medium 502, which includes a polishing surface 504. Medium 502 can include any of the polyurea-based material described herein, and can be used in connection with suitable polishing apparatus, which for illustration purposes can be similar to grinding apparatus 200, 300. Polishing surface 504 can include grooves of any suitable pattern. A bottom surface of portion 500 can include an adhesive. Exemplary polishing material includes up to 100 wt % polyurea-based material and preferably about 5 wt % to 80 wt % of the polyurea-based material as described herein.

The polyurea-based material can be used as a single pad or as a plurality of pads stacked on each other. For example, a stacked pad may comprise one or more pads as disclosed herein (i.e., including the polyurea-based material as described herein) as well as a typical pad (e.g., polyurethane, polyurea-polyurethane hybrid, or polyurea formed using a primary amine) or a plurality of pads as described herein without a typical polishing pad.

Methods of Forming Polyurea-Based Material and Media

Exemplary methods of forming a polyurea-based material include the steps of mixing one or more secondary polyamines having a general formula of R[—NH—R′]n, wherein n is greater than or equal to 2, wherein R is not H and wherein R′ is not H; reacting the secondary polyamine or polyamines with one or more of polyisocyanates, polyisocyanate derivatives, and polyisocyanate products to form a polyurea-based composition; and pouring the polyurea-based composition into a mold. The methods can include adding and mixing additional materials as described herein or as otherwise known in the art. Various polyurea-based materials as described herein can be prepared in the presence of a catalyst. Others can be formed without a catalyst. Classes of suitable catalysts include, but are not limited to, tertiary amines, such as triethylamine, and organometallic compounds, such as dibutyltin dilaurate.

The materials or components can be mixed together using, for example, high-shear blending to incorporate air and/or particles into a matrix. The polyurea-based material can be formed as a foam or matrix in an open mold.

Exemplary methods of forming polyurea-based material suitable for polishing or grinding media can be accomplished in a single mixer. In accordance with various exemplary methods, one or more secondary polyamines or derivatives or products thereof are mixed, for example, in an open-air container with the use of a high-shear impeller. During the mixing process, atmospheric air can be entrained in the mix by the action of the impeller, which pulls air into a vortex created by the rotation. The entrained gas bubbles can act as nucleation sites for a foaming process. A blowing agent, such as water, can be added to the mix to facilitate a reaction, which produces a gas, such as CO₂, resulting in cell growth. During this open-air mix and while in the liquid phase, other optional additives can be added to the mix, such as surfactants or additional blowing agents.

In addition to or in lieu of chemical foaming agents and cell openers, it may be possible to directly introduce gas bubbles into the mix during the mixing process. For example, while the mix is still in the liquid state, such as before the addition of polyisocyanates, or after the addition of polyisocyanates but within a low-viscosity window, or at any other suitable time, an output of a gas injector can be inserted directly into the open-air mix, causing injection of more bubbles than would otherwise be introduced through the action of the impeller alone. Optionally, one may apply micro-filtration to the output end of a pump, such as a gas injector pump, to promote the formation of very small bubbles, such as those in the 1-10 micron diameter range. A step of directly introducing gas bubbles can allow the selection of the size and quantity of bubbles.

In some example embodiments, there is a short time window after the addition of the one or more polyisocyanates or derivatives or products thereof of about 1-2 minutes, during which the viscosity of the mix remains low, called the “low-viscosity window.” The mix may be poured into a mold during this window. In one example embodiment, quickly after the pour, the window passes, and existing pores become effectively frozen in place. Although pore motion can essentially have ended, pore growth may continue, for example, as CO₂ continues to be produced from a polymerization reaction. In one example embodiment, the molds are oven cured, for example, for about 6 to about 12 hours at about 100° C. to about 130° C. or about 115° C. to substantially complete the polymerization reaction.

After oven curing, the molds can be removed from the oven, and allowed to cool. At this point, in the case of, for example, grinding media, the polyurea-based material can be machined (e.g., using a lathe or CNC tool) to produce a grinding wheel, such as tubular member 400.

Alternatively, the material can be skived to produce slices of the polyurea-based material that can be made into circular pads or rectangular-shaped pads or pads of any other desired shape. For example, the slices can be made by cutting to shape with a punch or cutting tool or any other suitable instrument. In some example embodiments, an adhesive is applied to one side of the pad. Additionally or alternatively, the polyurea-based material surface can be grooved, if desired, for example, on the polishing surface in a pattern, such as a cross-hatched pattern (or any other suitable pattern). By way of additional examples, a geometry or shape of grooves may comprise at least one of a square trough, a rounded trough, and a triangular trough. In addition to the specific embodiments disclosed, numerous physical configurations of various geometries to the polishing pad surface are contemplated in this disclosure. The grooves can be created via any mechanical method capable of producing grooves in a polyurea-based material as described herein. For example, grooves can be created with a circular saw blade, a punch, a needle, a drill, a laser, an air-jet, a water jet, or any other instrument capable of rendering grooves in the pad. Moreover, grooves can be made simultaneously with a multiple gang-saw jig, a multiple-drill bit jig, a multiple punch jig, a multiple-needle jig, or the like.

Although exemplary embodiments of the present disclosure are set forth herein, it should be appreciated that the disclosure is not so limited. For example, although materials and media are described with particular fillers, foaming agents, and the like, the disclosure is not necessarily limited to these examples. Various modifications, variations, and enhancements of the materials, methods, and media set forth herein may be made without departing from the spirit and scope of the present disclosure.

What is claimed is:

1. A polyurea-based material for grinding or polishing a surface of a substrate, the polyurea-based material comprising:
a polyurea, wherein the polyurea is formed from one or more secondary polyamines having a general formula of R[—NH—R]ₙ, wherein n is greater than or equal to 2 and wherein R is not H and R′ is not H; and one or more of polyisocyanates, polyisocyanate derivatives, and polyisocyanate products.

2. The polyurea-based material of claim 1, wherein the polyurea comprises a general formula of:

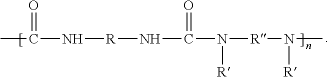

3. The polyurea-based material of claim 1, wherein a molecular weight of the polyurea ranges from about 50 to about 6000 Da.

4. The polyurea-based material of claim 1, wherein the polyurea-based material further comprises a cell stabilizer.

5. The polyurea-based material of claim 1, wherein the polyurea-based material further comprises from 0 wt % to about 80 wt % filler.

6. The polyurea-based material of claim 1, wherein the polyurea-based material comprises about 5 wt % to about 80 wt % of the polyurea.

7. The polyurea-based material of claim 1, wherein the polyurea-based material comprises grooves.

8. The polyurea-based material of claim 1, wherein the polyurea-based material comprises an adhesive on a surface.

9. A stacked pad comprising a polyurea-based material of claim 1.

10. A method of forming a polyurea-based material, the method comprising the steps of:
mixing one or more secondary polyamines having a general formula of R[—NH—R]ₙ, wherein n is greater than or equal to 2, wherein R is not H and wherein R′ is not H;
reacting the one or more secondary polyamines with one or more of polyisocyanates, polyisocyanate derivatives, and polyisocyanate products to form a polyurea-based composition; and pouring the a polyurea-based composition into a mold.

11. The method of forming a polyurea-based material of claim 10, further comprising a step of mixing one or more blowing agents with the one or more secondary polyamines.

12. The method of forming a polyurea-based material of claim 11, wherein the blowing agent is water.

13. The method of forming a polyurea-based material of claim 10, further comprising mixing one or more surfactants with the secondary polyamine.

14. The method of forming a polyurea-based material of claim 10, further comprising a step of curing the polyurea-based composition.

15. The method of forming a polyurea-based material of claim 10, further comprising a step of machining the polyurea-based material.

16. The method of forming a polyurea-based material of claim 10, further comprising a step of adding adhesive to a surface of the polyurea-based material.

17. The method of forming a polyurea-based material of claim 10, further comprising mixing a cell opener with the secondary polyamine.

18. The method of forming a polyurea-based material of claim 10, further comprising a step of directly introducing gas bubbles into a secondary polyamine mixture.

19. A polishing media comprising the polyurea-based material of claim 1.

20. A grinding media comprising the polyurea-based material of claim 1.
